(19) United States
(12) Patent Application Publication
(10) Pub. No.: US 2009/0171180 A1
(43) Pub. Date: Jul. 2, 2009

(54) METHOD AND APPARATUS FOR CONFIGURING WEARABLE SENSORS

(76) Inventors: Trevor Pering, San Francisco, CA (US); Roy Want, Los Altos, CA (US); Benjamin Kuris, Brookline, MA (US)

Correspondence Address: INTEL CORPORATION;c/o CPA Global, P.O. BOX 52050, MINNEAPOLIS, MN, 55402, US

(21) Appl. No.: 11/966,648

(22) Filed: Dec. 28, 2007

(51) Int. Cl. A61B 5/04

(52) U.S. Cl. 600/372

(57) ABSTRACT

A mobile device may use near field communication (NFC) technology to configuration a generic pool of wearable, wireless sensors that may occupy different physical locations on an end user s body. The wearable sensors have an embedded NFC tag in them such, that a mobile device, such as a mobile phone, with an NFC capability may be used to configure sensors in the field so that it recognizes which sensor is connected to which body part. Such a system may be used for example to monitor and report on the user's daily physical activity patterns or be used for video gaming applications where the user's physical movements are detected and utilized to control the game.

### FIELD OF THE INVENTION

Embodiments of the present invention relate to wearable sensors on various parts of the body and, more particularly, to configuration of wearable sensors by an end user.

### BACKGROUND INFORMATION

In many fields, for example in the health fields and the gaming arts, there may be a need for a person to wear a number of sensors on various parts of their body. In a health care setting, a doctor or technician may place and configure the sensors.

Such sensors may be used as wearable activity monitors. That is, devices that could monitor and report on the user's daily physical activity patterns. In some situations, a patient may need to wear several identical or similar sensors on many different parts of their body. For example, they may need to wear a sensor on both their left and right ankles. In this situation, it can be quite difficult to specify which sensor is located where. Furthermore, the user may have to don a large collection of sensors and it may be difficult or confusing as to where a specific sensor should be worn if they were already preconfigured to a particular location.

In a gamming environment, a network of wearable sensors attached, for example, to a user's arms and/or legs may enable a new class of physical game that would allow people to interact with the game. A racing game, for example, could be controlled by how fast somebody can shuffle their feet up and down, or arm and leg movements could control a fighting game. This capability would be similar to systems that use a wireless joystick to control a PC game with the additional benefit that the physical sensors would enable a more realistic gamming experience.

Currently, most wearable sensors must be manually configured when they are attached or they may be preconfigured for a particular body location. While these techniques may be acceptable for situations where a healthcare provider can configure or place the sensors, it is a significant hurdle that will make it difficult for the typical consumer to configure a wearable sensor system.

### BRIEF DESCRIPTION OF THE DRAWINGS

The foregoing and a better understanding of the present invention may become apparent from the following detailed description of arrangements and example embodiments and the claims when read in connection with the accompanying drawings, all forming a part of the disclosure of this invention. While the foregoing and following written and illustrated disclosure focuses on disclosing arrangements and example embodiments of the invention, it should be clearly understood that the same is by way of illustration and example only and the invention is not limited thereto.

FIG. 1 is a diagram illustrating an end user and a pool of generic wearable body sensors;

FIG. 2 is a diagram of an end user placing random generic sensors one various parts of their body;

FIG. 3 is a diagram of an end user configuring the location of the sensors with a mobile device;

FIG. 4 is a diagram of an end user having configured sensors on their body in communication with a mobile device; and

FIG. 5 is a flow diagram illustrating a method for configuring a plurality of generic body sensors.

### DETAILED DESCRIPTION

Described is a method and apparatus for configuring a system that uses wearable sensors facilitated by a mobile device with Near Field Communication (NFC) capability. NFC is an emerging technology that has been initially intended for point-of-sale interactions. For example, a smart-card or other device can act as a user's digital wallet which can be used to pay for a transaction at a point-of-sale terminal. NFC may be used as a technology for configuring wireless networks. For example, a user may scan the NFC component embedded in a wireless access point in order to configure their mobile device to securely establish a wireless connection. Using NFC in this way is advantageous because, it makes the configuration very easy to perform. That is, the user simply touches two devices together rather than requiring them to manually type in a lengthy association code, encryption key, or perform a laborious manual device discovery.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Embodiments use an NFC configuration capability and applies it to wireless sensors that may occupy different physical locations (as opposed to just virtual configuration, like a network ID). If the wearable sensors have an embedded NFC tag in them, a mobile device with an NFC reader may be used to configure the system so that it recognizes which sensor is connected to which body part. For example, the mobile device might prompt the user to scan the sensor on their left ankle, and then after that particular sensor has been registered to be associated with the left ankle, the user could then configure the system to record the next sensor attached to another body part.

Referring now to FIG. 1, there is shown a user 100 and a plurality is generic sensors 102, 104, 106, and 108. While there are four sensors shown in this example, one skilled in the art will understand that this is an arbitary number and in practice there may me more or fewer sensors. The sensors 102-108 may include accelerometers or other motion sense devices, temperature sensors, hear rate sensors, skin moisture sensors, and the like for monitoring the activity and physical state or similar health parameters of the wearer. In other embodiments, the sensors may be fashioned as jewelry, such as wrist watches, necklaces, anklets, earrings and the like so as not to be conspicuous.

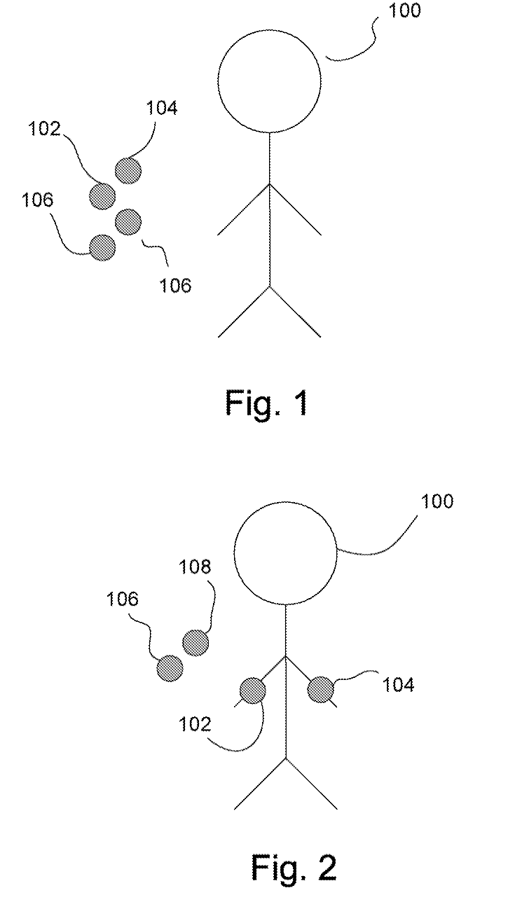

In FIG. 2, the user may attach random ones of the sensors, say sensor 102 and sensor 104, to their right and left arms, respectively. The remaining sensors may or may not get attached to their legs or other parts of the user's body 100.

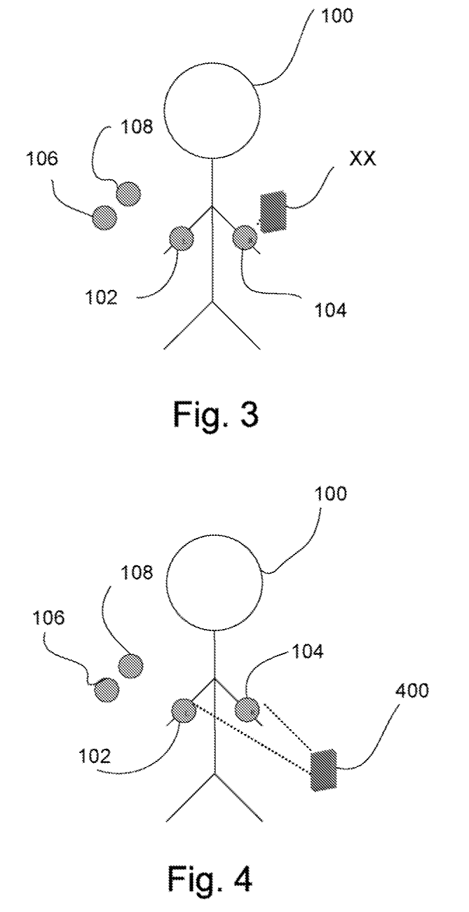

As shown in FIG. 3, a scanner device 300 with NFC capability may be used to configure a particular sensor to a particular part of the body. For example, the scanner 300 may prompt the user to touch or place the scanner 300 in near proximity to the sensor 104 that the user has placed on their right arm. The system would then know which of the generic sensors 102-108, was associated with the right arm, and so forth. This procedure may be repeated for each of the generic sensors 102-108. Alternately, the user 100 may place the sensor 104 on their right arm or other part of their body and thereafter touch or place the scanner 300 in near proximity to the sensor 104 and manually enter into the scanner 300 that this particular sensor 104 is associated with the right arm.

As shown in FIG. 4, a reader device 400 may be used to wirelessly link to the sensors 102 and 104, and there after be used to track movements or other parameters of the user's 100 right and left arms, respectively. The reader device 400 and the scanner device 300 may be the same device or may be two separate devices forming part of the same system. In one embodiment, the scanner and/or reader 400 may by the end user's mobile telephone, or personal digital assistant (PDA) device equipped with NFC capability.

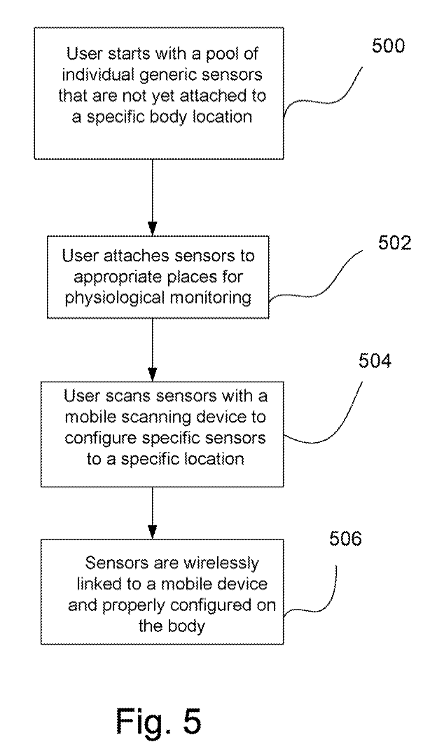

FIG. 5 shows a flow diagram illustrating the basic procedure for configuring a plurality of generic sensors 102-108. In block 500, the user starts with a generic pool of sensor devices. The sensors may all be the same and are not yet associated with any particular location in the body. In block 502, the user may attach random sensors to various locations on their body, such as arms and legs, head, etc. In block 504, the user scans the various sensors, one at a time, to configure the sensor to a specific location on the body. Finally, in block 506, the sensors may be wirelessly linked to a mobile device to track movements of the various parts of the body where the sensors are located.

This same basic technique can apply to a number of similar situations using NFC for disambiguation. Consider the case where someone has several wearable sensors that they rotate through to manage battery lifetime (i.e., use one sensor until the battery runs low, and then switch to a different sensor). This technique would allow the user to easily configure their mobile platform with new sensors while the old ones are being recharged. Furthermore, the scanning process could be used to trigger state changes in the sensors. For example, it may turn them “on” from a low-power sleep state. Using a sleep state may be advantageous since battery life is limited. Likewise, an NFC scan may also be used to trigger a data dump from the sensors. That is, the user or practitioner simply scans the wearable sensor with the mobile device and the sensor performs a data dump to the mobile device. Similarly, the mobile device may be used to scan and determine the state of the sensor (which may be too small or compact to have a significant display useful enough for communicating any diagnostic information).

Another valuable characteristic of using a personal mobile device, such as a mobile phone, to establish on-body associations with wearable sensors is to associate the sensors with a particular person through the association with their phone. Since the wearable sensors are small and don't have any significant I/O capability suitable for configuring the wearable sensor, it would be difficult to configure such a sensor in the field without a proximate IO capability to a mobile device with a full user interface (UI).

In another embodiment, the above described techniques may be used to configure sensors other than wearable sensors. For example, doors, appliances, and fixtures in the house may be fashioned with sensors to determine use. These systems may be used diagnostically to determine changes in psychological and physiological state of elders who may be suffering from Alzheimer's, or have limited mobility. Using the method described, it would be easier to deploy such a system and then later the NFC method could be used to map each sensor to its particular location and provide a label, for example, “kitchen door” or “stove control”, etc. The sensors may than be used to determine if an elder is using a door, window, cabinet door, or appliance.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various equivalent modifications are possible within the scope of the invention, as those skilled in the relevant art will recognize.

These modifications can be made to the invention in light of the above detailed description. The terms used in the following claims should not be construed to limit the invention to the specific embodiments disclosed in the specification and the claims. Rather, the scope of the invention is to be determined entirely by the following claims, which are to be construed in accordance with established doctrines of claim interpretation.

What is claimed is:

1. An system, comprising:
a plurality of wearable sensors to be worn anywhere on a user's body;
a near field communication (NFC) device associated with each of the wearable sensors; and
a mobile device including NFC capabilities to scan each of the sensors and associate an individual one of the plurality of sensors with a specific body part.

2. The system as recited in claim 1, wherein the mobile device comprises a mobile telephone.

3. The system as recited in claim 1 wherein the mobile device comprises a personal digital assistant (PDA).

4. The system as recited in claim 1 wherein the plurality of sensors are fashioned as jewelry.

5. The system as recited in claim 1 wherein the sensors includes motion sensors to monitor movement of the specific body part to which it is attached.

6. The system as recited in claim 5 wherein the sensors further comprise any of temperature sensors, hear rate sensors, skin moisture sensors.

7. The system as recited in claim 1 wherein the sensors can perform a data dump to the mobile device.

8. The system as recited in claim 1 wherein the mobile device can put anyone of the plurality of sensors into a sleep mode.

9. The system as recited in claim 4 wherein the jewelry comprises any of wrist watches, necklaces, anklets, and earrings.

10. A method, comprising:
providing a plurality of non-configured wearable sensors each having wireless capabilities;
donning individual ones of the plurality of non-configured sensors on particular parts of a body;
using a wireless device to scan individual sensors and configure each sensor to the particular part of the body; and
wirelessly communicating physiological information between the sensors and the wireless device.

11. The method as recited in claim 11 wherein the wireless capabilities comprises near field communication (NFC) technology.

12. The method as recited in claim 11 further comprising:
performing a data dump between ones of the sensors and the wireless device.

13. The method as recited in claim 11, further comprising:
fashioning the plurality of sensors to look like jewelry.

14. The method as recited in claim 13, wherein the jewelry comprises any of wrist watches, necklaces, anklets, and earrings.

15. The method as recited in claim 11 wherein the sensors comprise any of temperature sensors, hear rate sensors, skin moisture sensors.

16. The method as recited in claim 11 wherein the mobile device comprises a mobile telephone.

17. The method as recited in claim 1.1 wherein the mobile device comprises a personal digital assistant.

18. The method as recited in claim 11 further comprising.
using the wireless device to put a sensor into a sleep mode.

19. The method as recited in claim 10 further comprising:
using the physiological information to control a video game.

20. The method as recited in claim 11 further comprising:
using the physiological information to monitor health parameters of the user.
